(19) United States
(12) Patent Application Publication
(10) Pub. No.: US 2011/0123655 A1
(43) Pub. Date: May 26, 2011

(54) MULTI-PURPOSE INSECT REPELLANT COMPOSITION AND METHODS

(76) Inventor: Veronica Robinson, Carrington (AU)

(21) Appl. No.: 12/990,598

(22) PCT Filed: May 1, 2008
(86) PCT No.: PCT/US2008/062228

§ 371 Date: Feb. 4, 2011

(30) Foreign Application Priority Data
May 1, 2007 (AU) 76021100

(51) Int. Cl. A01N 65/22; A01P 7/04; A61K 36/53; A61P 17/02

(52) U.S. Cl. 424/745; 106/18.32

(57) ABSTRACT

Insect repelling compositions and methods of use are provided which include a composition containing neem oil, citronella oil, rosemary oil, an insecticide in an amount that is non-toxic, and a solvent or carrier, and its use in repelling insects. The composition can also include aromatic ingredients such as lemon grass oil, lulu scent, and peach oil, as well as other conventional ingredients such as emulsifiers, stabilizers, etc.. These compositions can be applied to a human or animal subject or to a non-living surface in order to repel insects safely and without side-effects, and can also be used to provide safe and effective treatment and healing of insect bites and wounds and other topical damage caused by insects such as burrowing insects.

### RELATED APPLICATIONS

The presently-disclosed subject matter claims the benefit of Australian Provisional Patent Application having the NPS Receipt No. 76021100 and the NPS Batch No. SPBI-07193219, filed May 1, 2007; the disclosure of which is incorporated herein by reference in its entirety.

### TECHNICAL FIELD

The presently-disclosed subject matter relates to compositions and methods for repelling insects. In particular, the presently-disclosed subject matter relates to compositions and methods for repelling insects wherein a composition comprising neem oil, citronella oil, rosemary oil, an insecticide, and a solvent or carrier is provided. The presently-disclosed subject matter further provides a method for treating insect bites using the compositions of the present invention.

### BACKGROUND

Insects, particularly head lice, ticks, mosquitoes, flies, and cockroaches are a perennial problem and are often regarded as pests to both animals and humans. Insects can carry many different diseases and their bites can cause itching and/or discomfort in both animals and humans and can also result in an infection of the bitten area. In addition, many humans and animals have a high sensitivity to the bites or infestations of various insects and may suffer serious allergic reactions and even death due to insects. Moreover, with regard to livestock and pets, insects can create havoc and insect-related problems which may have serious economic effects.

Consequently, compositions which repel or kill insects are commonly used to keep insects away from animals, humans, and non-living surfaces. However, many presently commercially available insecticides, including those available for human and/or household use, include harsh chemicals that can be present in concentrations that are toxic to humans and animals. In addition, many common insecticidal compositions are designed specifically for a particular type of insect and may not protect against a greater variety of insect pests.

In particular, various undesirable side effects have been observed after the use of commercially available insecticides. These side effects have included immediate or delayed neurotoxic reaction, suffocation, headaches, and/or nausea. Further, the harsh chemicals typically contained in currently available insecticides have been known to cause significant skin irritation when they are applied to a living surface, such as the skin of animals or humans.

Accordingly, there is an urgent need for improved compositions for repelling insects that provide safer and more effective alternatives to the present chemical-based insecticides. In addition, it would be highly desirable to develop safe and inexpensive insecticidal compositions which can aid against a variety of insects and which can be used not only to repel insects but to treat and heal insect bites and other wounds caused by insects.

### SUMMARY

This Summary includes several embodiments of the presently-disclosed subject matter, but is merely exemplary of the numerous and varied alternate embodiments which fall within the scope of the present invention. Accordingly, reference to one or more representative features of a given embodiment is likewise exemplary, and certain embodiments can typically exist with or without the feature(s) mentioned; likewise, those features can be applied to other embodiments of the presently-disclosed subject matter, whether listed in this Summary or not. In this regard, this Summary does not list or suggest all possible combinations of such features which fall within the scope of the invention.

In order to achieve the aforementioned objectives and to address the unmet needs outlined above, a composition for repelling insects is provided which in certain embodiments comprises a combination of ingredients including neem oil, citronella oil, rosemary oil, an insecticide in an amount that is non-toxic to humans and animals, and a solvent or carrier. In some of these embodiments, the composition can comprise about 1% to about 12% by volume neem oil, about 0.5% to about 10% by volume citronella oil, about 0.5% to about 10% by volume rosemary oil, and about 0.5% to about 6% of a safe and non-toxic insecticide, such as pyrethrum and permethrum, in a solvent or carrier such as paraffin oil or white oil. Further constituents of these compositions can include conventional ingredients, such as emulsifiers, stabilizers, etc., such as those types of conventional additives in other common topical insect repellant compositions.

Further, the composition for repelling insects may be prepared which comprises about 50 g/l to about 100 g/1 paraffin oil, about 80 g/l to about 140 g/l citronella oil, about 20 g/l to about 80 g/l rosemary oil, about 20 g/l to about 80 g/l neem oil, and an insecticide that is non-toxic to humans and animals in that range, in a solvent or carrier. Wherein the insecticide is pyrethrum or permethrum, the composition may include the insecticide in the range of about 10 g/l to about 40 g/l. The composition may further comprise conventional ingredients including emulsifiers, stabilizers, preservatives, thickeners, etc. In certain embodiments, additional components may include emulsifying wax, xanthan gum, and a preservative such as phenonip.

In certain embodiments of the presently-disclosed subject matter, the solvent or carrier utilized in the present composition may be any suitable solvent or carrier conventionally used in insecticidal formulations that are non-toxic to humans and animals. Suitable carriers or solvent may include paraffin oil, white oil, mineral oil, white mineral oil, baby oil, vegetable oil, and petroleum jelly. In other embodiments, the solvent or carrier may be a vegetable oil such as canola oil, olive oil, palm oil, soybean oil, pumpkin seed oil, corn oil, sunflower oil, safflower oil, peanut oil, grape seed oil, sesame oil, argan oil, and rice bran oil.

Additionally, the composition of the present disclosure may include a material to make the composition less oily, e.g., an ingredient such as litsea cubeba oil.

In additional embodiments, the present composition may further comprise an aromatic ingredient, for example, lemon grass oil, lulu scent, and peach oil. However, any suitable aromatic ingredients such as those currently used in insecticidal formulations may also be utilized in the present composition.

In certain additional embodiments of the presently-disclosed subject matter, a composition for repelling insects is provided wherein paraffin oil is used as the carrier, and is provided in an amount of about 1000 ml. In such embodiments, the present composition may include neem oil in an amount from about 10 ml to about 120 ml, citronella oil in an amount of about 5 ml to about 100 ml, and rosemary in an amount of about 10 ml to about 40 ml or rosemary oil is used. Further, these compositions may include about 5 ml to 60 ml of a non-toxic insecticide such as pyrethrum or permethrum.

The presently-disclosed subject matter further provides, in certain embodiments, an insecticide that is safe and non-toxic to humans and animals, and may comprise a suitable pediculicide. Suitable insecticides can include pyrethrum and permethrum. In these embodiments, the pyrethrum may comprise natural pyrethrum and the permethrum may comprise synthetic permethrum.

In accordance with the presently-disclosed subject matter, the present compositions may be used to repel a wide variety of insects, including those insects which are commonly found to affect human and animals, including head lice, ticks, mosquitoes, flies and cockroaches. The composition will generally be designed to kill or repel these and other insects, In other embodiments, a composition for repelling insects is provided wherein the composition kills insects.

Further provided, in some embodiments, is a composition for repelling insects wherein the composition treats insects bites or heals wounds caused by insects such as burrowing insects like ticks or lice. The present compositions can thus have both wound healing and insect repellant properties.

The compositions of the presently-disclosed subject matter may also be provided in any suitable form currently used to apply topical compositions to skin and hair. Such compositions may take the form of liquids, oils, foams, creams, lotions, pastes, sprays, or even a roll-on composition form.

The present composition may thus be used in methods of repelling insects and/or healing bites and wounds caused by biting and/or burrowing insects. In general, the present method for repelling insects comprises providing an insect repelling composition of the presently-disclosed subject matter and applying the composition to a living or non-living surface. In some embodiments, the surface is the skin or hair of a human. In some embodiments, the surface is the skin, hair, coat, or fur of an animal. Alternatively, the surface may be any suitable non-living surface which one desires to keep insect-free. Such surfaces may include any suitable household or outdoor surface including floors, walls, tables, barbecue areas, kennels, stables, gardens, sheds, and factories.

According to another aspect of the presently-disclosed subject matter, a method of treating insect bites and other wounds caused by insects is also provided. In these embodiments, the method comprises providing an effective amount of the composition of the presently-disclosed subject matter, and applying the composition topically to an insect bite or other wounds such as those caused by burrowing insects on a human or animal subject.

Accordingly, it is an object of the presently-disclosed subject matter to provide compositions and methods for repelling insects as well as methods of treating insect bites. This object is achieved in whole or in part by the presently-disclosed subject matter. Other objects and advantages will become evident to those of ordinary skill in the art by virtue of the following description of the presently-disclosed subject matter and non-limiting Examples.

### DETAILED DESCRIPTION

Unless defined otherwise, all terms used herein have the same meaning as commonly understood to one of ordinary skill in the art to which the presently-disclosed subject matter belongs. Although any compositions, methods, and materials similar or equivalent to those described herein can be used in the practice or testing of the presently-disclosed subject matter, representative compositions, methods, and materials are now described.

Following long-standing patent law convention, the terms “a”, “an”, and “the” refer to “one or more” when used in this application, including the claims. Thus, for example, reference to “an insecticide” or “a carrier” can include a plurality of such insecticides or carriers, respectively, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently-disclosed subject matter.

As used herein, the term “about,” when referring to a value or to an amount of mass, weight, time, volume, concentration or percentage would have its ordinary meaning to one skilled in the art and is meant to encompass variations as would be understood by one skilled in the art. Accordingly, in some embodiments this would be in the range of ±20% of the stated value, and in others would range from ±10% or lower, as would be recognized in the art as within the range necessary to perform the disclosed methods and achieve the results sought by the objectives as stated herein.

The presently disclosed subject matter thus provides compositions and methods for killing or repelling insects as well as methods of treating insect bites or other wounds such as those caused by insects. The term “repellant” or “repelling” is used herein to describe the deterring of insects from a particular area, individual or surface, and this term would be well understood by one skilled in the art. In general, the compositions of the disclosure can be used to deter insects from a given object and thus reduce the number of insects that may land or circle around a specific object, surface or individual, and thus reduce the likelihood of a bite or a wound caused to a human or animal, or reduce the number of insects approaching or landing on a particular surface. The compositions thus will be advantageous in that they will reduce the potential for harm and general nuisance or other serious problems caused by insects to humans and animals.

As used herein, it is recognized that the term “insect” will have its generally accepted meaning to one skilled in the art. In accordance with the disclosure, the present compositions can be used to repel or kill numerous insects including lice such as head lice, mosquitoes, cockroaches, flies, maggots, black flies, deer flies, horse flies, beetles, gnats, ticks, fleas, bed bugs, earwigs, ants, aphids, mites, and the like.

In some embodiments, a composition for repelling insects is provided that comprises neem oil, citronella oil, rosemary oil, an insecticide in an amount that is non-toxic to humans and animals, and a solvent or carrier. The insect repellant composition is thus comprised of a mixture of ingredients along with a suitable solvent or carrier. It is preferred that the solvent or carrier employed in the presently-disclosed compositions be ones that are safe and inert ingredients that have been conventionally used in other topical compositions that are harmless to humans and animals, and which do not affect the insect-repellant properties of the present composition.

Some examples of suitable carriers or solvent which can be employed in the present compositions for repelling insects include paraffin oil, white oil, mineral oil, white mineral oil, baby oil, vegetable oil, and petroleum jelly. In some embodiments, the solvent or carrier is vegetable oil that may be selected from the group consisting of canola oil, olive oil, palm oil, soybean oil, pumpkin seed oil, corn oil, sunflower oil safflower oil, peanut oil, grape seed oil, sesame oil, argan oil, and rice bran oil. In some embodiments, the solvent or carrier is paraffin oil which may be employed in an about 1000 ml.

The composition for repelling insects will thus include a combination of ingredients which, when combined, give an advantageous and effective means to repel or kill insects that may pose a nuisance or danger to humans and animals. In some embodiments these ingredients include, neem oil, extracted from the neem tree (Azadirachta indica), which is a long-lasting insect repellant. In the present compositions, neem oil may be used in amounts of from about 1% to about 12% of the volume of the composition, and more preferably will be used in a range of about 2% to about 6% of the volume of the composition. In a composition having a paraffin carrier in an amount of about 1000 ml, neem oil may be used in an amount from about 10 ml to about 120 ml, and more preferably from about 20 ml to about 60 ml.

The present composition will also include citronella oil. Citronella oil is known to those of skill in the art as an insect repellant and natural antiseptic; and can be obtained from the leaves and stems of different species of the Cymbopogon plant. For example, citronella oil can be obtained from Cymbopogon nardus. In the present composition, citronella oil may be present in an amount of about 0.5% to about 10% of the volume of the composition, and more preferably will be present in an amount of about 1% to about 4% of the volume of the composition. In a composition having a paraffin carrier in an amount of about 1000 ml, the citronella oil is used in an amount of from about 5 ml to about 100 ml, and more preferably from about 10 ml to about 40 ml.

The present composition will also include rosemary oil. Rosemary oil, obtained from Rosmarinus officinalis, is useful for its insect repelling properties, but also provides certain healing properties. Further, rosemary oil can also be useful in the treatment and prevention of head lice. In the present composition, rosemary oil may be present in an amount of about 0.5% to about 10% of the volume of the composition, and more preferably will be present in amounts of about 1% to about 4% of the volume of the composition. In a composition having a paraffin carrier in an amount of about 1000 ml, rosemary oil may be used in an amount of from about 5 ml to 100 ml, and more particularly may be used in an amount of about 10 ml to about 40 ml.

The present composition also preferably contains an insecticide which can be effective in an amount that is non-toxic to humans and animals. In the present composition, the insecticide may be included in the range of from about 0.5% to about 6% of the volume of the composition, but this will also depend on the nature and purpose of the composition as well as the particular insecticide that is chosen. More commonly, the insecticide will comprise about 1% to about 4% of the volume of the composition. In a composition according to this disclosure having a paraffin carrier in an amount of about 1000 ml, the insecticide may be used in an amount of from about 5 ml to about 60 ml, and more commonly will be used in an amount of from about 10 ml to about 40 ml.

Suitable insecticides in accordance with the disclosure will be those naturally occurring compound that repel or kill insects yet remain safe to humans and do not pose a problem with regard to side effects. For example, pyrethrum may be used as the insecticide in the composition of the present disclosure. Pyrethrum is the active constituent of the pyrethrum flower, and has been shown to be safe for use and has a rapid paralyzing effect on insects. In fact, pyrethrum has been considered by the United States Environmental Protection Agency as a safe an effective insecticide for humans and animals. Pyrethrum can also kill insects without being harmful to the environment as it is known to be biodegradable and easily broken down upon exposure to light. In the present composition, the pyrethrum used will preferably be the natural pyrethrum as described above, and a 50% concentrated form of pyrethrum may be used.

However, other suitable insecticides may be used such as chemically modified pyrethrums, such as permethrum, which are commercially available, have been deemed generally safe, and show greater persistence to breaking down. As such, in some embodiments, the present composition will include permethrum, such as synthetic permethrum

Even further, other compositions in accordance with the invention may be prepared wherein the concentrations of the ingredients will include about 80 g/l to about 140 g/l citronella oil, about 20 g/l to about 80 g/l rosemary oil, about 20 g/l to about 80 g/l neem oil, about 10 g/l to about 40 g/l of an insecticide that is non-toxic to humans and animals in that range, in a solvent or carrier, which may comprise about 50 g/l to about 100 g/l paraffin oil. Such ac composition can be prepared in about 2 liters of polyethylene glycol and 13 liters of water.

As would be recognized by one skilled in the art, the present compositions may be prepared by any conventional method wherein the aforementioned ingredients are combined to create a form suitable for topical application to skin or hair of humans, skin, hair or fur of an animal, or an appropriate living or non-living surface wherein repelling of insects is desired. In one suitable embodiment, ingredients including neem oil, rosemary oil, citronella oil and the suitable non-toxic insecticide are added into a carrier such as paraffin oil and mixed until a suitable consistency is achieved. Batches may be prepared using about 1000 ml of a carrier such as paraffin oil, and once all of the ingredients are combined and mixed, smaller amounts of the composition of the present disclosure may be placed in suitable containers for personal or consumer use, including bottles, tubes, spray cans, or other like consumer items which dispense similar topical products to skin or hair. In general, the compositions of the disclosure will comprise an effective amount of insect-repellant ingredients that is effective yet also non-toxic to humans and animals.

In addition, when preparing the present composition in the form of a cream, a suitable composition may be prepared in a process wherein a suitable amount of water, e.g., around 13 liters, is brought to a boil, then cooled to 65 ° C. Next, propylene glycol in a suitable amount, e.g., 2 liters is added, followed by a carrier such as white oil with an optional emulsifier such as emulsifying wax and then mixing. After this step, other optional ingredients such as preservatives (e.g., phenonip) and stabilizer/thickener (e.g., xanthan gum) may be added, and then the oils of the composition (neem oil, rosemary and citronella) can be added when the mixture is slightly cooled. Additional mixing and cooling of these ingredients will provide a cream form of the composition which is suitable as a roll-on cream.

As would be recognized by one skilled in this art, the present compositions can also include further ingredients such as emulsifiers, such as an emulsifying wax, stabilizers or thickeners, such as xanthan gum, preservatives, such as phenonip, and any other suitable ingredient that would be conventionally added to similar compositions. One skilled in the art would also recognize that the amounts and concentrations of these ingredients will vary from one composition to another depending on the nature and purpose of the particular composition. For example, suitable compositions may be prepared which contain about 10 to 40 g/l of an emulsifier such as emulsifying wax, about 1 to 10 g/l of a stabilizer or thickener such as xanthan gum, and about 5 to 20 g/l of a preservative such as phenonip.

The inventor has thus discovered that the present composition as described herein has surprisingly been found to have effective ability to repel and/or kill insects that are present while still providing a safe and effective composition that is non-toxic to humans and animals. The repelling or killing of insects, in accordance with the methods and compositions of the presently-disclosed subject matter, can be achieved by applying an effective amount of the compositions of the presently-disclosed subject matter. As used herein, the term “effective amount” can refer to an amount of the composition that is sufficient to repel or kill insects. As would be recognized by one skilled in the art, the exact amount that is required to be effective will vary from insect to insect and from subject to subject, depending on the species, age, and general condition of the insect or subject, the particular carrier being used, mode of administration, and the like. As such, the effective amount will vary based on the particular circumstances, and an appropriate effective amount can be determined in a particular case by one of ordinary skill in the art using only routine experimentation.

In another aspect of the invention, the present composition amy also include an aromatic ingredient. The addition of an aromatic ingredient is desirable in order to give the composition a pleasurable aroma or scent. As such, in some embodiments, the aromatic ingredient is selected from the group consisting of lemon grass oil, lulu scent, and peach oil.

Another advantage of the presently-disclosed subject matter is that, in some embodiments, additional ingredients may be added to reduce the oily texture of the insect repelling composition. In this regard, in some embodiments, the composition further comprises litsea cubeba oil.

The insect repelling composition of the presently-disclosed subject matter is also beneficial for both human and animal use in the treatment of insect bites and in healing wounds. For example, the inventor of the presently-disclosed subject matter has discovered that not only does the composition repel insects, but also soothes the skin of human or animals that has been previously bitten or is itching, and stops the itch in as little time as a few seconds. As another example, the insect repelling composition has been applied to flyblown ears of dogs and sheep and the wounds have healed, the maggots have been drawn out and killed, and the coat and wool has regrown. As such, in some embodiments, the composition treats insect bites and further has wound healing properties, such as those wounds resulting from burrowing insects. In some embodiments, a method for treating insect bites is provided that comprises providing a composition of the presently-disclosed subject matter and applying the composition to an insect bite on a human or animal subject.

As used herein, the terms “treatment” or “treating” would be considered to encompass these terms as used by one skilled in the art and would include any treatment of an insect bite or a wound caused by an insect bite, including, but not limited to, prophylactic treatment and therapeutic treatment As such, the terms treatment or treating include, but are not limited to: preventing an insect bite or a wound caused by an insect bite; inhibiting the progression of a wound caused by an insect bite; arresting or preventing the development of a wound caused by an insect bite; reducing the severity of an insect bite or a wound caused by an insect bite; ameliorating or relieving symptoms associated with an insect bite or a wound caused by an insect bite; and causing a regression of an insect bite or a wound caused by an insect bite or one or more of the symptoms associated with an insect bite or a wound caused by an insect bite.

The presently-disclosed compositions are preferably administered topically. As such, in some embodiments, of the presently disclosed subject matter the composition for repelling insects is in a form selected from the group consisting of liquids, oils, foams, cream, lotions, pastes, sprays, and roll-on compositions. Such formulations are known to those of ordinary skill in the art and may be chosen based on the surface to which the composition is to be applied and how the compositions are to be applied.

As indicated above, a roll-on cream for repelling insects, including mosquitoes, in accordance with the present composition may be prepared which has about 10 g/l to about 40 g/l emulsifying wax, about 60 g/l to about 80 g/l paraffin oil, about 100 g/l to about 120 g/l citronella oil, about 40 g/l to about 50 g/l rosemary oil, about 50 g/l to about 60 g/l neem oil, about 20 g/l to about 30 g/l of an insecticide selected from the group consisting of pyrethrum and permethrum, about 1 g/l to about 10 g/l xanthum gum, and about 5 g/l to about 20 g/l phenonip, in polypropylene glycol and water.

Further provided, in some embodiments of the presently disclosed subject matter, is a method for repelling insects. In some embodiments, the method for repelling insects comprises providing a composition of the presently-disclosed subject matter and applying the composition to a living or non-living surface. The inventor has further discovered that the presently-disclosed compositions are not only effective for human and animal use, but can also be used for repelling insects from non-living surfaces. As such, in some embodiments, the surface is the skin or hair of a human. In some embodiments, the surface is the skin, hair, coat or fur of an animal. Further, in some embodiments the surface is selected from the group consisting of floors, walls, tables, barbecue areas, kennels, stables, sheds, and factories.

Accordingly, the present invention can effectively and safely be used in the killing or repelling of insects, and to promote wound healing in situations such as where the wound is caused by insects such as burrowing insects.

The presently-disclosed subject matter is further illustrated by the following specific but non-limiting examples.

### EXAMPLES

Insect Repellant Oil

For general formulations, 1000 ml of paraffin oil may be added to a suitable container or beaker, and to the paraffin oil may be added 20 ml to 60 ml of neem oil, 10 ml to 40 ml of citronella oil, 10 ml to 40 ml of rosemary oil, and 10 ml to 20 ml of pyrethrum (50% concentrated) or 10 ml to 40 ml of synthetic permethrum. Depending on the desired aroma or scent, additional aromatic ingredients, such as lemon grass oil, lulu scent, or peach oil may be added to the mixture. To reduce the oily texture of the composition, litsea cubeba oil may be added until a desired texture is obtained.

Mosquito and Fly Repellant Spray

In a specific example, 10 ml of rosemary oil, 30 ml of citronella oil, 20 ml of neem oil, and 20 ml or pyrethrum or permethrum was mixed together with 920 ml of paraffin oil or vegetable oil so that total volume was about one liter. The composition was then placed in a spray container and used to spray humans and animals, as needed, to repel and kill insects. Once applied to the humans and animals, the composition stopped the itching of previous insect bites, and was shown to have healing and antiseptic properties. The application of the oil to animals killed flies, mosquitoes, fleas, and maggots, while healing wounds and enhancing the coat or fur of the animals. The oil had a long lasting effect and did not burn the skin of the animals.

The spray was also used as a surface spray for walls, floors, and windows. Upon spraying the composition onto these surfaces, insects such as spiders, ants, and cockroaches, as well as snails were killed and similar insects and animals were not seen in these areas for two to three months. Likewise, the spraying of the composition around the base of trees prevented caterpillar, and fruit fly infestation.

Lice Treatment Oil

In another specific example, 30 ml of rosemary oil, 30 ml of citronella oil, 35 ml of neem oil and 35 ml of pyrethrum or permethrum was added to 870 ml of different carriers including olive oil, paraffin oil, and vegetable oil to form a lice treatment oil. The lice treatment oil was then applied to the hair and scalp of a human. Upon application of the lice treatment oil, the lice were drawn out from under the surface skin of the scalp and killed. Additionally, the lice eggs were loosened from the hair shafts, and the scalp was soothed and healed by the application of the lice treatment oil.

Roll on Cream

To prepare a cream form of the composition suitable for use as a roll-on product, 13 liters of water was boiled and then subsequently cooled to 65° C. After cooling to 65° C., 2 liters of propylene glycol, 1050 g of paraffin oil, and 400 g of emulsifying wax was added to the water and mixed. 50 g of xanthum gum and 200 g of phenonip was then added and mixed into the composition. The mixture was then allowed to further cool and the oil ingredients of 1650 g of citronella oil, 650 g of rosemary oil, and 850 grams of neem oil were added to the composition. The composition was then mixed continuously and allowed to cool until a cream-like consistency was obtained.

The cream composition was then added to a container suitable for rolling-on the cream and was applied to a human subject. The cream effectively repelled flies, mosquitoes, and ticks from the human subjects, stopped itching from previous insect bites, and had healing and antiseptic properties.

It will be understood that various details of the presently-disclosed subject matter can be changed without departing from the scope of the subject matter. Furthermore, the foregoing description is for the purpose of illustration only, and not for the purpose of limitation.

1. A composition for repelling insects comprising neem oil, citronella oil, rosemary oil, an insecticide in an amount that is non-toxic in humans and animals, and a solvent or carrier.

2. The composition of claim 1, wherein the neem oil is present in an amount in the range comprises about 1% to about 12% of the volume of the composition.

3-9. (canceled)

10. The composition of claim 1, wherein the insecticide is selected from the group consisting of pyrethrum and permethrum.

11-19. (canceled)

20. The composition of claim 1, wherein the solvent or carrier is selected from the group consisting of paraffin oil, white oil, mineral oil, white mineral oil, baby oil, vegetable oil, and petroleum jelly.

21. The composition of claim 20, wherein the solvent or carrier is a vegetable oil selected from the group consisting of canola oil, olive oil, palm oil, soybean oil, pumpkin seed oil, corn oil, sunflower oil, safflower oil, peanut oil, grape seed oil, sesame oil, argan oil and rice bran oil.

22-29. (canceled)

30. The composition of claim 1, further comprising litsea cubeba oil.

31. The composition of claim 1, wherein the insecticide is a pediculicide.

32. The composition of claim 1, wherein said composition is in a form selected from the group consisting of liquids, oils, foams, creams, lotions, pastes, sprays and roll-on compositions.

33. A method of repelling insects comprising providing the composition according to claim 1, and applying the composition to a living or non-living surface.

34. (canceled)

35. The method of claim 33, wherein the surface is the skin, hair, coat or fur of a human or an animal.

36. The method of claim 33, wherein the surface is selected from the group consisting of floors, walls, tables, barbecue areas, kennels, stables, gardens, sheds, and factories.

37. The method of claim 33, wherein the insects are selected from the group consisting of head lice, flies, and cockroaches.

38-42. (canceled)

43. A method of treating insect bites or other wounds caused by insects comprising
providing the composition according to claim 1, and
applying the composition to an insect bite on a human or animal subject.

44. A composition for repelling insects comprising about 4% to about 6% by volume neem oil, about 1% to about 4% by volume citronella oil, about 1% to about 4% by volume rosemary oil, and about 1% to about 4% of an insecticide selected from the group consisting of pyrethrum and permethrum, in a paraffin oil carrier.

45. A composition for repelling insects comprising about 50 g/l to about 100 g/l paraffin oil, about 80 g/l to about 140 g/l citronella oil, about 20 g/l to about 80 g/l rosemary oil, about 20 g/l to about 80 g/l neem oil, about 10 g/l to about 40 g/l of an insecticide that is non-toxic to humans and animals in that range, in a solvent or carrier.

46. The composition of claim 45 wherein the solvent or carrier comprises polypropylene glycol and water.

47. The composition of claim 45 wherein the insecticide is selected from the group consisting of pyrethrum and permethrum.

48. The composition of claim 45 further comprising a material selected from the group consisting of emulsifiers, stabilizers, preservatives, thickeners, and mixtures thereof.

49. The composition of claim 45, wherein the composition is in a form selected from the group consisting of creams, lotions, pastes, sprays, and roll-on compositions.

50. The composition of claim 45, wherein the composition further comprised a material selected from the group consisting of an emulsifying wax, xanthan gum, phenonip, and mixtures thereof.
